(19) United States
(12) Patent Application Publication
(10) Pub. No.: US 2024/0197215 A1
(43) Pub. Date: Jun. 20, 2024

(54) OPTOELECTRONIC APPARATUS FOR AND METHOD OF MEASURING ORGANIC TISSUE

(71) Applicant: OULUN YLIOPISTO, Oulu (FI)

(72) Inventor: Jan NISSINEN, Oulu (FI)

(21) Appl. No.: 18/557,540

(22) PCT Filed: Apr. 25, 2022
(86) PCT No.: PCT/EP2022/060853

§ 371 Date: Oct. 26, 2023

(30) Foreign Application Priority Data
Apr. 27, 2021 (FI) 20215487

(51) Int. Cl. A61B 5/1455; A61B 5/00

(52) U.S. Cl. CPC A61B 5/14552; A61B 5/6813; A61B 2562/0238

(57) ABSTRACT

An optoelectronic apparatus for measuring organic tissue is attached with the organic tissue. A semiconductor optic radiation source outputs repeatedly infrared pulses of toward the tissue. An array of single-photon avalanche diodes is directed toward the tissue and detects photons of the optical pulses that have interacted with the tissue. A timing unit determines time-of-flights of photons of each of the optical pulses within a temporal measurement range after an output of each of the optical pulses. A data processing unit estimates a physiological state of the tissue within at least one time window shorter than the temporal measurement range based on at least one of the following: a number of the detections within the time window and a distribution of the detections within the time window.

### FIELD

The invention relates to an optoelectronic apparatus for and a method of measuring organic tissue.

### BACKGROUND

Oxygen content variation of blood and pulse of the heart can be measured optically in a non-invasive manner using a transmissive oximeter. The oximeter attached to a fingertip or earlobe typically has a continuously illuminating LED (Light Emitting Diode), although a laser may also be used, that outputs infrared light, and the oximeter measures absorbance of the light that depends on oxygenation of haemoglobin. A reflectance oximeter performs the measurement basically in a similar manner to the transmissive measurement except that the light does not travel from one side of a section of body such as chest or arm to another side but instead light scattered and/or reflected back from the section of a body is measured. These measurements work, however, poorly for skin, muscles and venous blood, for example.

Time-domain near infrared spectroscopy, which is based on transmission and reception of short optical pulses and determination of temporal duration therebetween, has also been used for providing concentration values of oxygenation levels of haemoglobin. However, the devices are not fully developed. Hence, improvement in biological and/or bio-signal measurements performed in an optical manner would be welcome.

### BRIEF DESCRIPTION

The present invention seeks to provide an improvement in the measurements.

The invention is defined by the independent claims. Embodiments are defined in the dependent claims.

### LIST OF DRAWINGS

Example embodiments of the present invention are described below, by way of example only, with reference to the accompanying drawings, in which

FIG. 1 illustrates an example of a time-of-flight measuring apparatus;

FIG. 2 illustrates an example of a measurement of organic tissue;

FIG. 3 illustrates an example of a measurement with optical cables;

FIG. 4 illustrates an example of a coaxial measurement;

FIG. 5 illustrates an example of an array of single-photon avalanche diodes (SPADs);

FIG. 6 illustrates examples of output and receive optical pulses;

FIG. 7 illustrates an example of separation of useful signal from disturbance;

FIG. 8 illustrates an example of the SPADs a part of which is covered by a polarizer;

FIG. 9 illustrates an example of a change of slopes of haemoglobin and lactate concentrations;

FIGS. 10 and 11 illustrate examples of the measuring apparatus of separate parts that communicate through radio transmission;

FIG. 12 illustrates an example of a data processing unit;

FIG. 13 illustrates an example of a single circuit board; and

FIG. 14 illustrates of an example of a flow chart of a measuring method.

### DESCRIPTION OF EMBODIMENTS

The following embodiments are only examples. Although the specification may refer to “an” embodiment in several locations, this does not necessarily mean that each such reference is to the same embodiment(s), or that the feature only applies to a single embodiment. Single features of different embodiments may also be combined to provide other embodiments. Furthermore, words “comprising” and “including” should be understood as not limiting the described embodiments to consist of only those features that have been mentioned and such embodiments may also contain features/structures that have not been specifically mentioned. All combinations of the embodiments are considered possible if their combination does not lead to structural or logical contradiction.

It should be noted that while Figures illustrate various embodiments, they are simplified diagrams that only show some structures and/or functional entities. The connections shown in the Figures may refer to logical or physical connections. It is apparent to a person skilled in the art that the described apparatus may also comprise other functions and structures than those described in Figures and text. It should be appreciated that details of some functions, structures, and the signalling used for measurement and/or controlling are irrelevant to the actual invention. Therefore, they need not be discussed in more detail here.

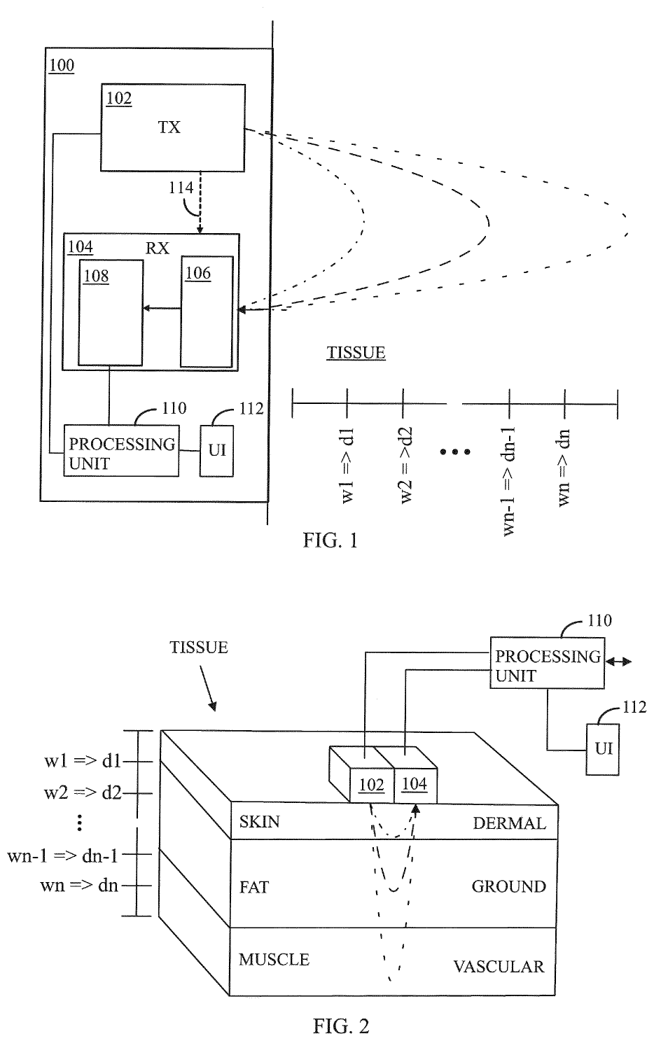

FIG. 1 illustrates an example of an optoelectronic apparatus 100 utilizing time-of-flights of photons of optical pulses. A semiconductor optic radiation source 102 outputs repeatedly infrared pulses. In an embodiment, the semiconductor optic radiation source 102 may comprise a CMOS (Complementary Metal Oxide Semiconductor) radiation source. The optic radiation source 102 may comprise one or more optically radiating semiconductor lasers. A plurality of the semiconductor lasers may be in a form of an array.

In an embodiment, duration of the optical pulses may be less than about 1 ns, for example. In an embodiment, duration of the optical pulses may be less than about 500 ps, for example.

The repetition of the optical pulses may be regular or irregular. The repetition may have a certain frequency, for example. The repetition rate may be adjustable.

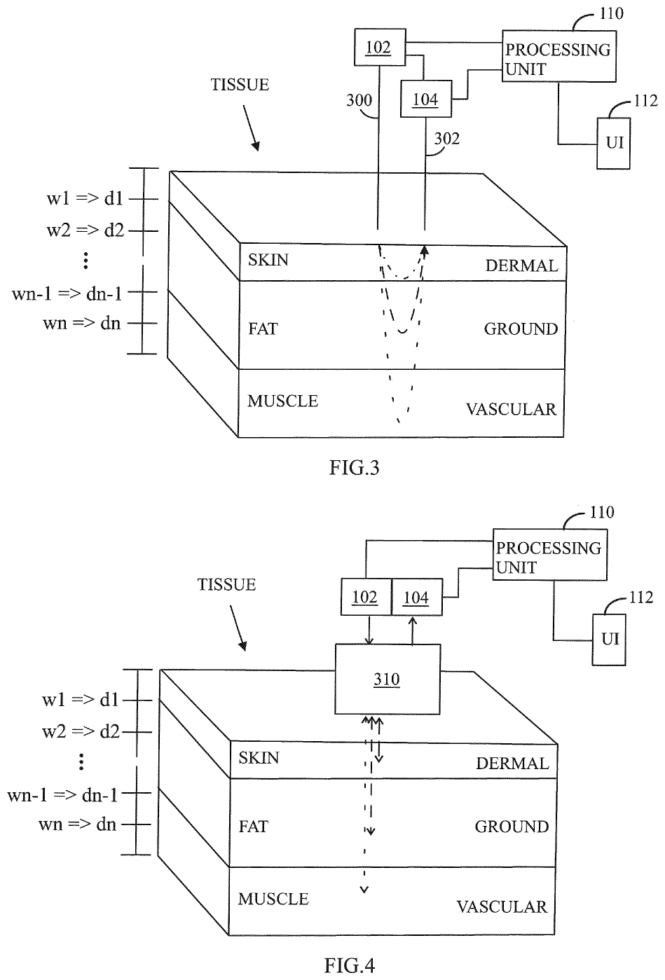

The optical pulses are directed toward an organic tissue, which is illustrated in more detail in FIGS. 2, 3 and 4. In an embodiment, the organic tissue may be a piece of meat, for example. The organic tissue may be living tissue. In an embodiment, the organic tissue may be that of an animal, for example. In an embodiment, the organic tissue may be that of a mammal such as a human being, for example. The organic tissue of a living animal can be considered to have three basic layers from surface towards inside: skin, fat and muscle as illustrated in FIGS. 2 and 3. In an embodiment, the optoelectronic apparatus 100 may be worn by the animal such as the human being.

In an embodiment, the organic tissue may be that of a plant such as a tree, grass or moss, for example. The organic tissue of a plant can be considered to have three basic layer from surface towards inside: dermal, ground and vascular as illustrated in FIGS. 2, 3 and 4. However, the number of layers is may be different or the layers may be unimportant for the measurement presented in this document.

The organic tissue may include one organ or a plurality of organs. Tissue of a single organ is a community of similar cells that function in similar manner. Blood is both tissue of cells and flowable substance. The physiological state of blood may refer to a level of haemoglobin, tendency of the level of haemoglobin and/or difference between oxidized haemoglobin and deoxidized haemoglobin. Additionally or alternatively the physiological state of blood may refer to variation of blood volume as a function of time in the tissue i.e. pulsation of the heart. Correspondingly, a physiological state of a plant tissue may depend on at least one flowable substance therewithin.

An array 106 of single-photon avalanche diodes 400 of an optical receiver 104, an example of which is illustrated in FIG. 4, is also directed toward the tissue. That the single-photon avalanche diodes 400 are directed toward the organic tissue means that field-of-views 402 of the single-photon avalanche diodes 400 are directed toward the organic tissue. The single-photon avalanche diodes 400 are configured to detect photons of the optical pulses that have interacted with the organic tissue.

The single-photon avalanche diodes (SPADs) 400 of the optical receiver 104 operate in the Geiger mode and not in a linear mode. Hence, the output of the single-photon avalanche diodes 400 is binary indicating only a detection or non-detection, the detection being triggered by a single photon.

FIG. 2 illustrates an example of a measurement where the radiation source 102 and the optical receiver 104 are attached with the skin or the dermal layer.

FIG. 3 illustrates an example where optical transmission cable 300 and optical reception cable 302 are used. The optical cables 300, 302 may comprise one or more optical fibers. In FIG. 3, the optical cables 300, 302 are side by side but they could also be coaxial. It is also possible that only the radiation source 102 has the optical cable 300 and the optical receiver 104 without the optical receiving cable 302 is in contact with the skin or the dermal layer. Alternatively, it is possible that only the optical receiver 104 has the optical receiving cable 302 and the radiation source 102 without the optical transmission cable 300 is in contact with the skin or the dermal layer.

A timing unit 108, which comprise an electric circuit, determines a time interval between an output of an optical pulse and reception of each of the photons of the optical pulse from the tissue. In this application, the term “determine” in its various grammatical forms may mean calculating, computing, data processing for deriving a result, looking up in a database or the like. As a result “determine” may also mean select, choose or the like.

The optical pulse transmitted from the the radiation source 102 may travel along various paths through the tissue, which are illustrated with dashed lines in FIG. 1 to 4. The the radiation source 102 and the optical receiver 104 may be connected such that with an optical pulse transmitted towards the tissue, a reference signal 114 is simultaneously transmitted to the optical receiver 104 for determining an output moment of an optical pulse. A portion of the optical pulse may be transmitted to the optical receiver 104 via a partially reflective mirror or prism or the like as the reference signal 114 for determining the output moment of an optical pulse, while a major portion of the optical pulse is transmitted towards the tissue. Alternatively, the the radiation source 102 may directly transmit another electrical or optical pulse as the reference signal 114 to the optical receiver 104 for determining the output moment. In general, the reference signal 114 has a determined temporal dependence with respect to the optical pulse transmitted towards the tissue for determining the time-of-flights.

A portion of the photons of an output optical pulse 500 reflect and/or scatter from the tissue and hit the array 106 of the single-photon detectors 400. In any of the single-photon detectors 400, the photon may be detected as a result of a high-speed breakdown in the detector 400. The timing jitter of the detection may be in a CMOS single-photon detector at the level of about 10 ps to 50 ps. Moreover, the breakdown may introduce immediately a logic level signal (e.g. 3 V) and thus no analog amplifiers may be needed. In addition to the 2D array 106 of the SPAD detectors 400, the optical receiver 104 may also include the timing unit 108, which may have a plurality of time-to-digital converters (TDCs). In general, the timing unit 108 may be a part of the optical receiver 104 or it may be outside of the optical receiver 104.

In an embodiment, the semiconductor optic radiation source 102 may enable the array 106 of single-photon avalanche diodes 400 for detections but it may be the array 106 of single-photon avalanche diodes 400 that controls the time-to-digital converters. That is, the time-to-digital converter consumes electric energy only in conjunction with the detections of photons at the single-photon avalanche diodes 400 which save energy in general. The length of the temporal measurement range 510 may be determined using a delay from the transmission of the optical pulse. After this determined delay detections are stopped. The temporal measurement range 510 may be based on a temporal range of the time-to-digital converter.

Time-to-digital conversion may thus be realized with about 10 ps to 50 ps resolution, for example, and it may measure the arrival-times of detected photons for reconstructing distribution of time of flight of photons.

The functionality of the time-to-digital converters is thus to measure the interval between the emitted laser pulse 500 and the introduced breakdown in every SPAD detector 400 that detected a photon. These intervals are the transit times of the photons from the radiation source 102 to the optical receiver 104 via the organic tissue.

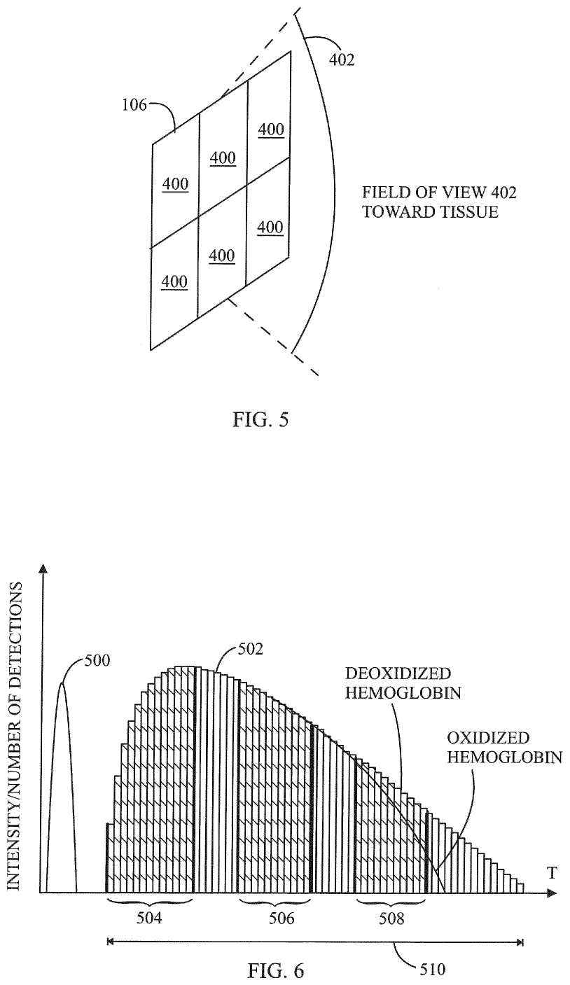

FIG. 5 illustrates examples of the output optical pulse 500 and the received optical pulse 502 interacted with the organic tissue. The x-axis is time T in an arbitrary scale and the y-axis is intensity and number of detections in arbitrary scales. The timing unit 108 determines time-of-flights of photons of each of the optical pulses within a temporal measurement range 510 after an output of each of the optical pulses. The temporal range 510, which is an allowed range within which time-of-flights of photons are measured, may start after a certain dead-time from an output of an optical pulse such that a reflection from the optical components of the apparatus, hair(s), other extension and/or liquid(s) layer or drops on the outer surface and/or the outer surface of the tissue itself are cut out from the measurement, for example. Each of the time-of-flights is based on an output moment of an optical pulse from the semiconductor optic radiation source 102 and detection moments of photons of the optical pulse 502 by the single-photon avalanche diodes 400 of the array 106.

Because of scattering in the tissue the received optical pulse 502 is much wider than the output optical pulse 500. A data processing unit 110 estimates or determines a physiological state of the tissue within at least one time window 504, 506, 508 shorter than the temporal measurement range 510 based on at least one of the following: a number of the detections within each of the time window 504, 506, 508 and a distribution of the detections within at least one time window 504, 506, 508. In this manner, the organic tissue may be measured in a layered manner regardless layered or non-layered structure of the organic tissue. Here, the term “estimate” in its various grammatical forms may mean calculate, compute, data process, look up in a database or the like for at least approximate result.

The physiological state may define or estimate functions of the at least one organ or at least one part of the at least one organ of the organic tissue. A variety of changes in the organic tissue result in changes in transit times of photons which may make the shape of the optical pulse 502 interacted with the organic tissue depend on the change in the tissue. That is, the transit times of the photons are a function of a state of the organic tissue. The shape refers to distribution of detections over time. Alternatively or additionally, a number of detections at a certain moment or within a time window may vary as a function of state of the organic tissue.

In an embodiment, each of the single-photon avalanche diodes 400 may detect a single photon of each of the optical pulses that is scattered from the tissue toward the array 106.

In an embodiment, the array 106 of single-photon avalanche diodes 400 may detect at least one of the following: a number of detections of at least one wavelength dominantly absorbed by blood with deoxidized hemoglobin; and a number of detections of at least one wavelength dominantly absorbed by blood with oxidized hemoglobin. The deoxidized hemoglobin mainly resides in veins. The deoxidized hemoglobin is within blood that is non-saturated with oxygen. The oxidized hemoglobin mainly resides in arteries. The oxidized hemoglobin is within blood that is saturated with oxygen.

FIG. 6 illustrates an example of how deoxidized hemoglobin and oxidized hemoglobin affect a shape of optical pulse and/or the number detections. The y-axis denotes the intensity of the optical pulse 500 and the number of detections of the received optical pulse 502 in arbitrary scales, and the x-axis denotes time T in an arbitrary scale. FIG. 6 is partially a histogram where the received pulse 502 is illustrated as bars. In time scale, a width of each bar represents an integration period over which detections are counted for one optical pulse. For a more reliable measurement, a sum of the number of detections of a plurality of optical pulses may be counted for each bar (that has been done in the example of FIG. 6). The integration period of a single bar may be determined be a temporal resolution of the time-to-digital conversion, for example. The integration period may thus be about 10 ps to 50 ps i.e. a temporal width of each of the bars, for example. However, some other integration period may also be used. Any of the bars alone may be used as a time window within which a measurement are performed. However, a measurement may be performed over a plurality of bars like in time windows 504, 506 and 508, for example.

The time window 504 may related to skin or dermal. The time window 506 may relate to fat or ground. The time window 508 where the time-of-flight is longer than in the time windows 504 and 506 can be considered to relate to muscle of an animal. The more oxidized the hemoglobin of blood of the muscle is, the lower is the number detections in the time window 508 relating to the muscle. The difference between the oxidized hemoglobin of blood of the muscle and the deoxidized hemoglobin of blood can also be seen the shape of the received optical pulse 502. Namely, the received optical pulse 502 is the shorter (see the continuous line), the more oxidized hemoglobin of blood is in the muscle. Corresponding changes may be observed in plants.

An optical photoplethysmogram (PPG) measurement can be used to detect variation of blood volume in the tissue based on changes in light absorption. In an embodiment of this document, the time-of-flights of photons of each of the optical pulses may be utilized to detect variation of blood volume such that a number of detections photon in at least one time window is determined. The number of detections is related to the absorption of the photoplethysmogram measurement. In an embodiment, a measurement rate of the variation of blood volume may be about ten measurements per second. In an embodiment, a measurement rate of the variation of blood volume may be about five measurements per second.

A received optical signal X_{dk}(t) as function of time t from a certain depth dk of the tissue may be expressed in a mathematical form in the following manner:

X_{dk}(t)=H_{dk}(t)+D_{dk}(t)

where H_{dk}(t) is a temporal variation of strength i.e. number of detections caused by the variation of blood volume i.e. pulsation of heart in the depth dk, and D_{dk}(t) includes other temporal variation such as optical losses as a function of time and movement of the tissue which can be considered disturbance. The movement of the tissue may be caused by movement of body or limb, for example.

The received optical signal X_{dn}(t) as function of time from depths d1 to dn of the tissue may be expressed in a mathematical form in the form of group of equations:

$${X}_{d 1} ( t ) = {H}_{d 1} ( t ) + {D}_{d 1} ( t ) \\ {X}_{d 2} ( t ) = {H}_{d 2} ( t ) + {D}_{d 2} ( t ) \\ ⋮ \\ {X}_{dn} ( t ) = {H}_{dn} ( t ) + {D}_{dn} ( t )$$

which may be expressed as a matrix:

$${X}_{d} ( t ) = {H}_{d} ( t ) + {D}_{d} ( t )$$

where X_{d}(t), H_{d}(t) and D_{d}(t) are time dependent matrices.

There is one to one correspondence between the depths d1 to dn and time windows w₁ to wₙ of measured time-of-flights. The time windows w₁ to wₙ can also be considered to be ranges of times of flights. That is, the data processing unit 110 may perform an estimation of the physiological state of the tissue within each of a plurality of the time windows as a function of time. The disturbance D_{dk}(t) varies as a function of depth and thus of the time window because different levels of the tissue respond differently to movement and contact variation, but the variation of blood volume i.e. each phase of the pulsation of heart occurs synchronously in all depths and time windows. As shown in FIG. 7, this makes it is possible for the data processing unit 110 to cancel out or reduce the disturbance and to separate and determine the variation of blood volume i.e. pulsation of heart in a reliable manner also under disturbance based on the estimation. The pulsation of the heart which may also be called heartbeat may be separated from the received signal using principal component analysis (PCA), independent component analysis (ICA), Kalman filter, digital filter, neural network, artificial intelligence, machine learning or the like, for example. A person skilled in the art is familiar with disturbance cancellation, reduction and/or separation of a useful signal, per se.

As explained, the data processing unit 110 estimates the physiological state at at least one unique depth range 504, 506, 508, w1 to wn within the tissue using detected photons that have interacted with the tissue inside at least one time window 504, 506, 508, w1 to wn shorter than the temporal measurement range 510. Each of the at least one time window 504, 506, 508, w1 to wn corresponds to a unique depth range d1 to dn within the tissue. The data processing unit 110 can then estimate the physiological state at at least one unique depth range 504, 506, 508, w1 to wn based on a number of the detections within each of the at least one time window 504, 506, 508, w1 to wn. The data processing unit 110 can alternatively estimate the physiological state based on a distribution of the detections of a plurality of time windows 504, 506, 508, w1 to wn. Of course, a combination of these is also possible.

FIG. 8 illustrates an example where a part of the SPADs 400 of the array 106 is covered by a polarizer 800. The polarizer 800 may be used to give more information on the tissue.

The data processing unit 110 may then estimate or determine physiological state related to lactic acid. In an embodiment, the data processing unit 110 may estimate or determine build-up of lactic acid in the tissue, for example.

In an embodiment, the data processing unit 110 may perform the estimation based on the number of detections of the at least one wavelength dominantly absorbed by the blood with oxidized hemoglobin. In an embodiment, the data processing unit 110 may perform the estimation based on difference between the number of detections of the at least one wavelength dominantly absorbed by the blood with deoxidized hemoglobin and the number of detections of the at least one wavelength dominantly absorbed by the blood with oxidized hemoglobin. In an embodiment, the data processing unit 110 may perform the estimation based on the number of detections of the at least one wavelength dominantly absorbed by the blood with oxidized hemoglobin, and difference between the a number of detections of the at least one wavelength dominantly absorbed by the blood with deoxidized hemoglobin and the number of detections of the at least one wavelength dominantly absorbed by the blood with oxidized hemoglobin. In an embodiment, at least one of these measurements may be performed from a muscle.

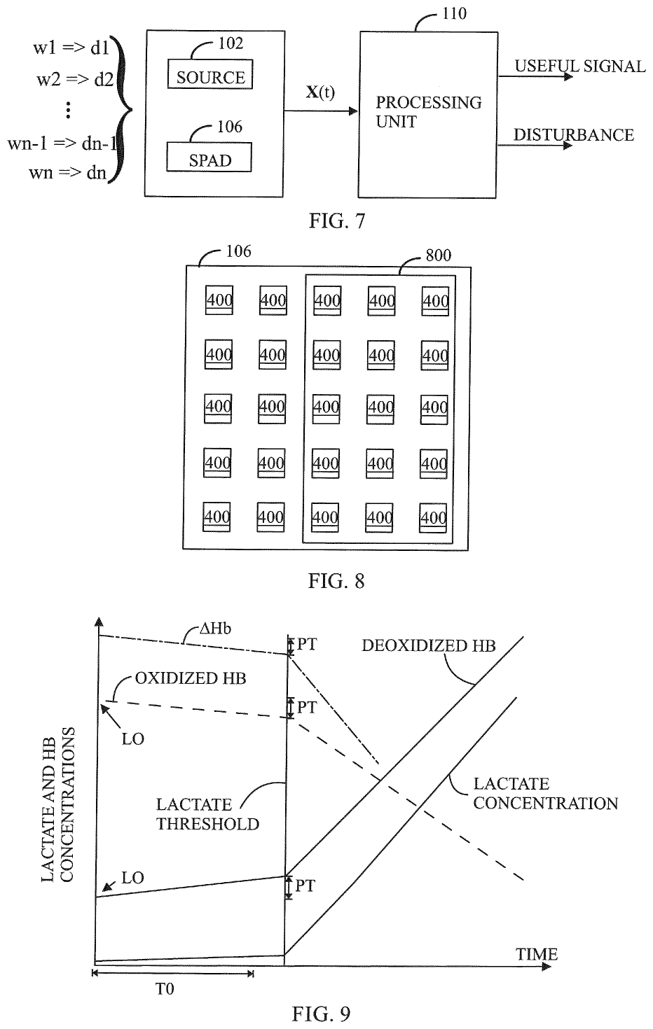

FIG. 9 illustrates an example of tendency of how lactate concentration, oxidized haemoglobin and deoxidized haemoglobin concentrations behave in a muscle tissue during incremental exercise from rest to maximal load. The vertical axis denotes lactate and haemoglobin concentrations in arbitrary scale. The horizontal axis denotes time in arbitrary scale.

In order to delivery enough oxygen to the working muscles for the aerobic production of adenosine triphosphate (ATP) molecules in a high intensity activity, the respiratory rate of the athlete increases. If the load of the training is high, the oxygen delivery to the muscles is too slow to produce the adequate amount of energy the muscle requires. In that case the body's energy production shifts to the anaerobic condition at a threshold (see FIG. 9). In this condition, energy production is replaced by lactic acid fermentation. Thus, measuring the oxidation of haemoglobin (oxidized haemoglobin Hb02 and/or deoxidized haemoglobin Hb) as explained above, the moment of the shift to anaerobic condition and the lactate threshold of the specific muscle can be determined.

During the incremental exercise, the oxidized haemoglobin Hb02 will decrease slowly before the time point of blood lactate threshold and then decrease. The decrease may be radical and potentially linear. Lactate of the blood increases slowly before the occurrence of lactate threshold and then increases. The increase may be sharp and potentially linear. Hence, there is a correlation between measured oxidized haemoglobin Hb02 and lactate threshold.

In an embodiment, approach to the turning point of lactate production i.e. the lactate threshold may be determined by evaluating the change in the slope of the oxidized haemoglobin Hb02 and/or the deoxidized haemoglobin Hb in blood. As the decrease of the oxidized haemoglobin Hb02 and/or the increase of the deoxidized haemoglobin Hb in blood has continued for a predetermined time T0 and/or change of the oxidized haemoglobin Hb02 and/or the deoxidized haemoglobin Hb with respect to the original level LO is equal to or larger than a predetermined change PT. In this manner, physical exercise may be lightened or stopped before the detection that the lactate production is at the lactate threshold. The predetermined time T0 and the predetermined change PT may be adaptive. The predetermined time T0 and the predetermined change PT may vary with respect to sex, exercise type, exercise length, age, physical condition, amount of physical training earlier or the like, for example.

In an embodiment, the turning point to the lactate production i.e. the lactate threshold may be determined by evaluating how rapid the change in the slope of the oxidized haemoglobin Hb02 and/or the deoxidized haemoglobin Hb in blood. In an embodiment, when the slope is different at different moments, the lactate threshold is between the moments of time. In an embodiment, when difference of the slope at different moments is larger than a predetermined value PV, the lactate threshold may be considered to be between said moments of time. In this manner, physical exercise may be lightened or stopped at the detection that the lactate production has reached at the lactate threshold. The predetermined value PV may vary with respect to sex, exercise type, exercise length, age, physical condition, amount of physical training earlier (professional, intensive amateur, normal amateur/exerciser, occasional exerciser, non-exerciser, for example) or the like, for example.

The difference ΔHb of the oxidized haemoglobin Hb02 and the deoxidized haemoglobin Hb also shows similar turning point and the variation tendency to lactate of the blood. Consistent with the physiology theory, the curve of difference ΔHb will show a fluctuation period around the analyzed oxygen dissociation point. Rightly in this fluctuation period, the metabolism status of the body is changed. The difference ΔHb may be a function of the oxidized haemoglobin Hb02 and the deoxidized haemoglobin Hb such as their difference. Alternatively or additionally, the difference ΔHb may be a function of a change of the oxidized haemoglobin Hb02 and a change of the deoxidized haemoglobin Hb.

In an embodiment, the data processing unit 110 may determine temporal variation of detections of a plurality of output optical pulses 500, the detections being within a window 506 of a middle part of the optical pulse 502 which is received after interaction with the tissue that is assumed fat tissue when examining an animal. The data processing unit 110 may then compensate the temporal variation of the detections in a time window 508 that has longer time-of-flights than the detections of the window 506 of the middle part of the optical pulse 502 based on the temporal variation of detections within the window 506 of the middle part of the optical pulse 502 which is received after interaction with the tissue that is fat tissue. The fat tissue has less circulation of blood than other tissues and the fat tissue is less prone to disturbances coming from the tissue related to the time window 508 deeper below the skin, which allows the data processing unit 110 to determine and reduce effect of noise and systematic disturbance.

Correspondingly, ground tissue of a plant may be used to determine temporal variation of detections of a plurality of output optical pulses 500. And in a similar manner the ground tissue of the plant is less prone to disturbances coming from the tissue related to the time window 508 deeper below the dermal, which allows the data processing unit 110 to determine and reduce effect of noise and systematic disturbance.

Noise and the systematic disturbance may be caused by movement of the animal or the plant.

In an embodiment, at least one of the timing unit 108 and data processing unit 110 may be programmable such that at least one of the temporal measurement range 510 and the at least one time window 504, 506, 508 is repeatedly adjustable. The at least one time window 504, 506, 508 may be adjusted manually or automatically. Manual adjustment may be performed through a user interface 112. In this manner, the organic tissue may be scanned in a depth direction, for example.

In an embodiment, the semiconductor optic radiation source 102 and the array 106 of single-photon avalanche diodes 400 may be spaced apart by a non-zero distance on the skin, an example of which is illustrated in FIG. 2.

In an embodiment an example of which is illustrated in FIG. 4, an output section 310 of the semiconductor optic radiation source 102 and an input section 310 of the array 104 of single-photon avalanche diodes 400 may be optically coaxial on the skin. In this embodiment, optical radiation hits the same area of the skin or the dermal both when entering the animal or the plant and when exiting the animal or the plant.

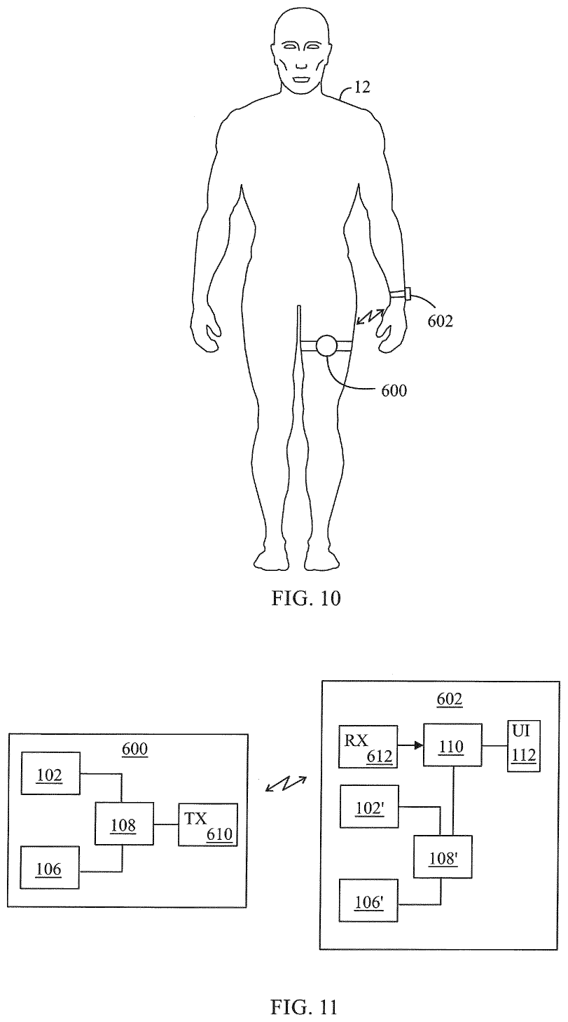

In an embodiment an example of which is illustrated in FIGS. 10 and 11, the optoelectronic apparatus 100 may comprise separately a muscle measurement device 600, which is wearable and attached to a muscular area of a body 12 of an animal such as a human being. The muscular area may be a thigh or upper arm, calf, neck, for example. The muscle measurement device 600 may comprise a radio transmitter 610, the semiconductor optic radiation source 102, the array 106 of single-photon avalanche diodes 400 and the timing unit 108. The optoelectronic apparatus may also comprise a separate wearable device 602, which comprises a radio receiver 612 and the data processing unit 110. The separate wearable device 602 may be attached at a wrist, for example. The radio transmitter 610 may transmit information on detections to the radio receiver 612, which may feed the information to the data processing unit 110 for determining physiological state of the tissue at the muscular area of the body 12. The user interface 112 may present the results to a user or a person or a personal performing the examination. The radio transmitter 610 and the radio receiver 612 may perform transmissions using electromagnetic radiation of radio frequency, Bluetooth®, WLAN (Wireless Local Area Network) or the like.

In an embodiment, the separate wearable device 602 may comprise an additional semiconductor optic radiation source 102′, and an additional array 106′ of the single-photon avalanche diodes 400 for determining physiological state of the tissue at the wrist. The additional semiconductor optic radiation source 102′, and the additional array 106′ of the single-photon avalanche diodes 400 are similar to the semiconductor optic radiation source 102, and the array 106, respectively.

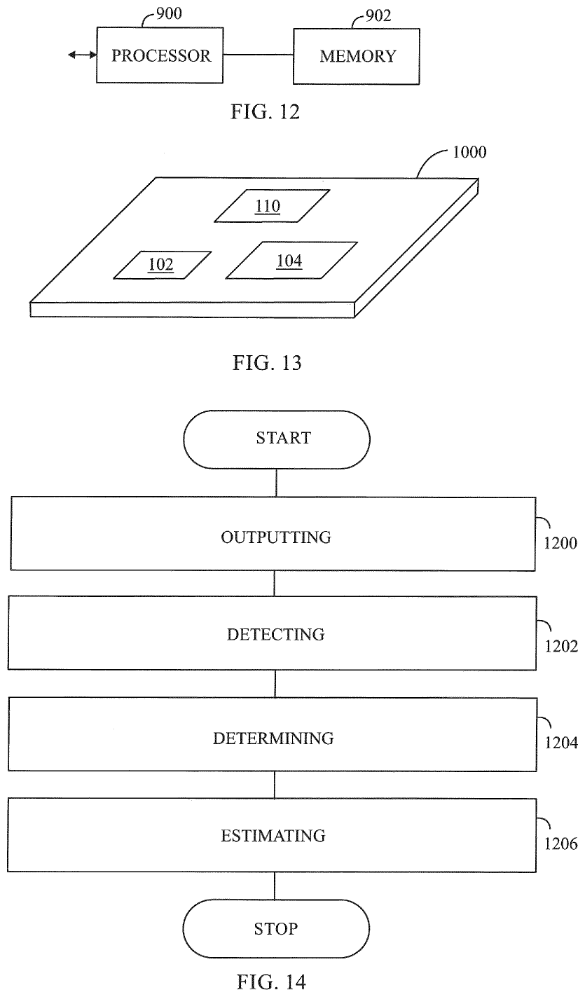

In an embodiment an example of which is illustrated in FIG. 12, the data processing unit 110 may comprise one or more processors 900, and one or more memories 902 including computer program code. The one or more memories 902 and the computer program code may, with the one or more processors 900, cause the data processing unit 110 at least to estimate the physiological state of the tissue.

FIG. 13 illustrates an example of a single circuit board 1000 the optoelectronic apparatus for measuring organic tissue. The circuit board 1000 may comprise the semiconductor optic radiation source 102, the array 106 of single-photon avalanche diodes 400 and the data processing unit 110. By integrating the electronic circuits on the single circuit board the optoelectronic apparatus can be made wearable. In in embodiment, the weight of the optoelectronic apparatus can be kept at or below 100 g by also properly selecting the packaging and case material. In in embodiment, the weight of the optoelectronic apparatus can be kept at or below 50 g by properly selecting the packaging and case material. In in embodiment, the weight of the optoelectronic apparatus can be kept at or below 10 g.

In the manner explained, it is possible to form a histogram based on timings of detections of photons scattered from various and/or determined depths of the tissue. Namely, the depth can be determined by selecting or setting a suitable time-window. From these histograms i.e. digital time-of-flights it is possible to separate several bio-signals and/or unintended movement (disturbance) of the tissue which are mixed together but which appear and affect differently in different depths. Because of the differences depending of depths the separation is possible. FIG. 14 is a flow chart of the measurement method. In step 1200, infrared pulses is output repeatedly toward the tissue by a semiconductor optic radiation source 102.

In step 1202, photons of the optical pulses that have interacted with the tissue are detected by an array 106 of single-photon avalanche diodes 400 which is directed toward the tissue.

In step 1204, time-of-flights of photons of each of the optical pulses within a temporal measurement range after an output of each of the optical pulses are determined by a timing unit 108.

In step 1206 a physiological state at at least one unique depth range within the tissue is estimated using detected photons that have interacted with the tissue inside at least one time window 504, 506, 508, w1 to wn shorter than the temporal measurement range 510, by a data processing unit 110, where each of the at least one time window (504, 506, 508, w1 to wn) corresponds to a unique depth range (d1 to dn) within the tissue, the estimation being based on at least one of the following: a number of the detections within the at least one time window 504, 506, 508, w1 to wn and a distribution of the detections within the at least one time window 504, 506, 508, w1 to wn.

The method of estimating a physiological state of the tissue of FIG. 13 may be implemented as a logic circuit solution or computer program. The computer program may be placed on a computer program distribution means for the distribution thereof. The computer program distribution means is readable by a data processing device, and it encodes the computer program commands, carries out the measurements and optionally controls the processes on the basis of the measurements.

The computer program may be distributed using a distribution medium which may be any medium readable by the controller. The medium may be a program storage medium, a memory, a software distribution package, or a compressed software package. In some cases, the distribution may be performed using at least one of the following: a near field communication signal, a short distance signal, and a telecommunications signal.

It will be obvious to a person skilled in the art that, as technology advances, the inventive concept can be implemented in various ways. The invention and its embodiments are not limited to the example embodiments described above but may vary within the scope of the claims.

1. An optoelectronic apparatus for measuring organic tissue, wherein the optoelectronic apparatus, which is attached with the organic tissue, comprises:
a semiconductor optic radiation source, which is configured to output repeatedly infrared pulses of toward the tissue;
an array of single-photon avalanche diodes, which is directed toward the tissue and which is configured to detect photons of the optical pulses that have interacted with the tissue;
a timing unit, which is configured to determine time-of-flights of photons of each of the optical pulses within a temporal measurement range after an output of each of the optical pulses;
a data processing unit, which is configured to estimate a physiological state at at least one unique depth range within the tissue using detected photons that have interacted with the tissue inside at least one time window shorter than the temporal measurement range, where each of the at least one time window is configured to correspond to a unique depth range within the tissue, the estimation being based on at least one of the following: a number of the detections within each of the at least one time window and a distribution of the detections of a plurality of time windows.

2. The apparatus of claim 1, wherein each of the single-photon avalanche diodes is configured to detect a single photon of each of the optical pulses that is scattered from the tissue toward the array.

3. The apparatus of claim 1, wherein the array of single-photon avalanche diodes is configured to detect at least one of the following: a number of detections of at least one wavelength dominantly absorbed by blood with deoxidized hemoglobin and a number of detections of at least one wavelength dominantly absorbed by blood with oxidized hemoglobin; and
the data processing unit is configured to estimate the physiological state related to lactic acid based on at least one of the following:
the number of detections of the at least one wavelength dominantly absorbed by the blood with oxidized hemoglobin, and
difference between the number of detections of the at least one wavelength dominantly absorbed by the blood with deoxidized hemoglobin and the number of detections of the at least one wavelength dominantly absorbed by the blood with oxidized hemoglobin.

4. The apparatus of claim 1, wherein the data processing unit is configured to perform the estimation of the physiological state of the tissue within each of a plurality of the time windows as a function of time, and separate and determine pulsation of heart based on the estimation.

5. The apparatus of claim 1, wherein at least one of the timing circuit and data processing unit is programmable such that at least one of the temporal measurement range and the at least one time window is repeatedly adjustable.

6. The apparatus of claim 1, wherein the semiconductor optic radiation source and the array of single-photon avalanche diodes are spaced apart by a non-zero distance.

7. The apparatus of claim 1, wherein an output section of the semiconductor optic radiation source and an input section of the array of single-photon avalanche diodes are optically coaxial.

8. The apparatus of claim 1, wherein the optoelectronic apparatus comprises a muscle measurement device, which is attached to a muscular area of a body and comprises a radio transmitter, the semiconductor optic radiation source, the array of single-photon avalanche diodes and the timing circuit, and a separate wearable device, which comprises a radio receiver and the data processing unit; and the radio transmitter is configured to transmit information on detections to the radio receiver, which is configured to feed the information to the data processing unit for determining the physiological state of the tissue at the muscular area of the body.

9. The apparatus of claim 8, wherein the separate wearable device comprises an additional semiconductor optic radiation source, and an additional array of the single-photon avalanche diodes for determining the physiological state of the tissue.

10. The apparatus of claim 1, wherein the data processing unit comprises one or more processors, and one or more memories including computer program code; and
the one or more memories and the computer program code configured to, with the one or more processors, cause the data processing unit at least to estimate the physiological state of the tissue.

11. A method of measuring organic tissue, the method comprising
outputting repeatedly, by a semiconductor optic radiation source, infrared pulses toward the tissue;
detecting photons of the optical pulses that have interacted with the tissue by an array of single-photon avalanche diodes which is directed toward the tissue;
determining, by a timing unit, time-of-flights of photons of each of the optical pulses within a temporal measurement range after an output of each of the optical pulses;
estimating, by a data processing unit, a physiological state at at least one unique depth range within the tissue using detected photons that have interacted with the tissue inside at least one time window shorter than the temporal measurement range, where each of the at least one time window corresponds to a unique depth range within the tissue, the estimation being based on at least one of the following: a number of the detections within the at least one time window and a distribution of the detections within the at least one time window.

12. The method of claim 11, the method further comprising detecting, by the array of single-photon avalanche diodes, at least one of the following: a number of detections of at least one wavelength dominantly absorbed by blood with deoxidized hemoglobin and a number of detections of at least one wavelength dominantly absorbed by blood with oxidized hemoglobin; and
estimating, by the data processing unit, the physiological state related to lactic acid based on at least one of the following:
the number of detections of the at least one wavelength dominantly absorbed by the blood with oxidized hemoglobin, and
difference between the number of detections of the at least one wavelength dominantly absorbed by the blood with deoxidized hemoglobin and the number of detections of the at least one wavelength dominantly absorbed by the blood with oxidized hemoglobin.

13. The method of claim 11, characterized by performing, by the data processing unit, an estimation of the physiological state of the tissue within each of a plurality of the time windows as a function of time, and
separating and determining pulsation of the heart based on the estimation.
